SEQUENCE LISTING (6 sequences)

SEQ ID NO: 1 - protein, 6 aa, Human peptide
  MOD_RES (1)..(1): FORMYLATION, Nle
XLFXYL

SEQ ID NO: 2 - protein, 6 aa, Human peptide
  MOD_RES (1)..(1): Nle
XLFXYK

SEQ ID NO: 3 - DNA, 21 bp, Human DNA
gtctccagttggactagccac

SEQ ID NO: 4 - DNA, 21 bp, Human DNA
aatgtcctcccatggccttcc

SEQ ID NO: 5 - DNA, 25 bp, Human DNA
gctctggctgtgcattcagcagatt

SEQ ID NO: 6 - DNA, 25 bp, Human DNA
aaaagtcagccagggccaggttacg

(19) United States
(12) Patent Application Publication
(10) Pub. No.: US 2006/0171944 A1
(43) Pub. Date: Aug. 3, 2006

(54) N-formyl peptide receptor mediation of platelet chemotaxis toward injured cells and activation of immune response

(76) Inventors: Julie A. Lekstrom-Himes, Gaithersburg, MD (US); Allan D. Kirk, Potomac, MD (US); Meggan Czapiga, Washington, DC (US); David E. Kleiner, Rockville, MD (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 10/263,791

(22) Filed: Oct. 4, 2002

(51) Int. Cl. A61K 39/395; A61K 38/17

(52) U.S. Cl. 424/143.1; 514/12

(57) ABSTRACT

The present invention relates to N-formyl peptides that induce an immune response, specifically, stimulating mobilization of platelets to a site of injury, thereby healing wounds. Further, the invention relates to N-formyl peptide receptor inhibitors, specifically N-formyl peptide receptor antibodies, that inhibit an immune response, thereby blocking inflammation and the mobilization of platelets and other phagocytic cells to a site of injury.

### FIELD OF THE INVENTION

This invention relates to N-formyl peptides and methods of their use in the mobilization of platelets and other phagocytic cells (monocytes, macrophages, neutrophils) to an injury site and activation of immune response. Further, the invention relates to the inhibition of inflammation at an injury site by administering N-formyl peptide receptor inhibitors, such as, for example, antibodies.

### BACKGROUND OF THE INVENTION

There are currently two prevailing models explaining how adaptive immune responses are initiated, self-nonself discrimination and the danger model, and both recognize a critical, yet ill-defined, inductive role of innate immunity^{1,2,3,4,5,6,7}. In both models naive T cell activation proceeds as a result of T cell antigen receptor (TCR) engagement accompanied by additional costimulatory signals provided by antigen presenting cells (APC, generally monocytes, macrophages and dendritic cells). In the self-nonself discrimination model it has been suggested that APC detect the presence of potential pathogens using pattern recognition receptors specific for evolutionarily conserved bacterial and fungal motifs^{5,8}. This provides the signals for APC activation needed for them to supply the costimulation necessary for the initiation of immunity. Thus the recognition of “fundamental foreignness” by the innate immune system is combined with the recognition of “specific foreignness” by T cell antigen receptors^{5,8}. In contrast, the danger model predicts that costimulation is derived from APC that have been activated by adjacent cell stress or other indicators of tissue damage independent of the presence of a foreign pathogen^{1,2,3,4}. It further predicts that in the absence of these signals, TCR engagement leads to acquired tolerance9,10,11,12. Endogenous agents, released by necrotic cells, activate APC and, in the presence of antigen, act as a natural adjuvant, inducing a primary immune response^{9,13,14}.

In theory, danger signals are endogenous cellular components normally sequestered from immune surveillance that are released with cell injury, suffering, or death, and are detectable by APCs. A variety of proposed danger signals, including unmethylated CpG sequences¹⁵, reactive oxygen species¹⁶, and heat shock proteins^{17,18,19} has been shown to activate APCs. Furthermore, APC activation has also been shown to be fundamentally associated with the engagement of the APC-based molecule CD40²⁰. Binding of CD40 to its ligand CD154 (also known as CD40 ligand) results in IL-12 production, eliciting T cell interferon-γ production, costimulatory molecule up-regulation (such as CD80), heightened phagocytic activity and antigen presentation, thus fostering the differentiation of naive T-cells into effector cells^{21,22,23,24,25}. Hence, regardless of the model, the cross linking of CD40 by CD154 provides the key signals in the induction of T cell mediated immunity. Indeed, treatment with neutralizing antibody specific for CD154 can effectively shut down the alloimmune response to the point of rendering recipients of mismatched allogeneic organ transplants tolerant to the organs^{10,11}.

It has recently been shown that in addition to activated T-cells, thrombin-activated platelets are a source of CD154^{26,27}. This is surprising given the traditionally limited view of platelets as agents in clotting and thrombus formation and suggests a novel role for platelets in providing a primal danger signal, one not requiring previous APC or T cell activation. It furthermore suggests a fundamental link between the body's homeostatic response to trauma and activation of an adaptive immune response that is independent of foreign pathogens. Such a link is consistent with the multi-system organ failure caused by dysregulated immune activation following severe trauma, the rejection of transplanted organs, and the lack of response seen in most tumors. Application of this function to platelets as a critical initiator of immunity, however, depends upon a reliable mechanism of directing platelet migration to sites of tissue damage, beyond the accepted stochastic mechanism of platelet-endothelial adherence. When cells die in a non-programmed fashion, sequestered mitochondrial N-formyl peptides may be released and recognized by platelets via functional N-formyl peptide receptors. Furthermore, endogenous N-formyl peptides derived and released from the mitochondria of necrotic cells may elicit platelet chemotaxis and provide CD154 to tissue-based APC as an endogenous danger signal linking acquired immune activation to trauma. Conversely, cells dying a programmed or apoptotic death may sequester N-formyl peptide thus avoiding immune activation from physiological cell turnover.

Formyl peptides are short peptides generated by bacterial or mitochondrial endopeptidase cleavage of the first few amino acids including the N-formyl-modified methionine group of proteins²⁸. They bind to specific receptors on phagocytic cells, and induce directed migration or chemotaxis^{29,30,31}. Human phagocytes express two N-formyl peptide receptors, FPR (N-formyl peptide receptor) and FPRL 1 (FPR-like 1), both of which couple to pertussis toxin-sensitive G proteins^{31,32,33}. FPR binds N-formyl peptides at a 1,000 fold higher affinity than FPRL 1 and is attributed with inducing chemotaxis^{31,32}. Based on their chemotactic actions, it has been hypothesized that N-formyl peptides attract phagocytes to sites of infection and injury and therefore may play an important role in microbicidal and other host defense activities^{34,35}.

### SUMMARY OF THE INVENTION

This invention relates, in general, to N-formyl peptides or variations of peptides. In addition, the invention relates to methods of using the N-formyl peptides or derivatives thereof for stimulating an immune or inflammatory response. Further, methods of using N-formyl peptide receptor inhibitors, such as blocking antibodies or other receptor antagonists, for inhibiting inflammation.

It is an object of the present invention to provide a pharmaceutical composition comprising N-formyl peptides, or variants thereof, and a carrier.

It is a further object of the present invention to provide a pharmaceutical composition comprising N-formyl peptide receptor antibodies, or variants thereof, and a carrier.

It is another object of the invention to provide a method of mobilizing platelets at an injury site comprising administering an effective amount of N-formyl peptides to mobilize the platelets.

It is a further object of the invention to provide a method of wound healing at an injury site comprising administering an effective amount of N-formyl peptides to heal a wound or accelerate aspects of wound healing.

It is also an object of the invention to provide a method of inhibiting inflammation, particularly counter adaptive immune responses such as autoimmunity or organ allograft or xenograft rejection, at an injury or engraftment site by administering an effective amount of N-formyl peptide receptor antagonists such as for example antibodies or other antagonists.

### BRIEF DESCRIPTION OF THE DRAWING

FIG. 1 shows human platelets that express N-formyl peptide receptors. a, Flow cytometric analysis of N-formyl peptide binding on human platelets, treated with 0, 0.001, 0.1 or 10 U thrombin and either unstained (upper panel) or stained with 10⁻⁷ M flourescein conjugated N-formylated peptide; fNLFNYK-fluorscein (middle panel) or stained with 10⁻⁷ M flourescein conjugated non-N-formylated peptide; NLFNYK-fluorscein (bottom panel). b and c, Confocal microscopy imaging of non-permeablized human platelets treated with 0 U or 10 U thrombin and stained with 10⁻⁶ M fNLFNYK-fl. d and e, Confocal microscopy imaging of permeablized human platelets treated with 0 U or 10 U thrombin and stained with 10⁻⁶ M fNLFNYK-fl. f and g, Confocal microscopy imaging of permeablized human platelets treated with 1 U thrombin and stained with 10⁻⁶ M fNLFNYK-fl or 10⁻⁶ M non N-formyl NLFNYK-fl. Differential interference contrast (DIC) image to confirmed the presence of platelets in panel G (insert).

FIG. 2 shows expression of formyl peptide receptor (FPR) and formyl peptide receptor-like 1 (FPRL 1) by reverse transcriptase PCR and expression of FPR protein by immunoblotting with FPR specific antibody. a, Expression of formyl peptide receptor and formyl FPRL 1 by reverse transcriptase PCR. MEG-01 cells were either left untreated or treated with 10⁻⁷ M PMA for 5 days to induce megakaryocyte differentiation and platelet production. The HL-60 cell line was used as a positive control for expression FPR and FPRL 1. The constitutively expressed “housekeeping” gene β-actin was used as a reference gene for normalization. b, Expression of FPR by immunoblotting extracts from platelets (lane 3), FPRL1 transfected HEK 293 cells (lane 2) and differentiated CD34+ cells (lane 1) probed with purified mouse anti-human monoclonal antibody. Immunoblot was stained with mouse anti-human β-actin monoclonal antibody (lower panel) to assess sample loading.

FIG. 3 shows calcium flux in activated and non-activated human platelets induced by N-formyl peptides. Human platelets were pretreated with 0 U or 1 U thrombin, loaded with fura-2 and stimulated with a 1 U thrombin, b 10⁻⁶ M fMLF, or C 10⁻⁶ M non-formylMLF. Representative traces from at least four independent experiments are shown.

FIG. 4 shows chemotaxis of human platelets in response to N-formyl peptides. a, Human platelets treated with 0 U (dashed line) or 10 U thrombin (solid line) were seeded upon a two micron pore membrane and allowed to migrate towards the indicated dose of the N-formyl peptide fMLF placed in the lower chamber of a transwell dish. Thrombin-activated platelets showed significantly more migration compared with non-activated platelets. b, Platelet chemotaxis is dependent upon the presence of an N-terminal formyl group. Thrombin-activated platelets showed significantly more migration toward fNLFNYK-fl compared with noN-formyl peptide NLFNYK-fl at both 10⁻⁶ and 10⁻⁷ M peptides. c, Platelet chemotaxis was blocked by mouse anti-human monoclonal antibody to FPR (α-FPR). Platelet treatment with 100 μg α-FPR prevented chemotaxis to fMLF compared with platelets treated with 100 μg isotype-specific antibody, IgG. d, Platelet migration was inhibited by pertussis toxin. Platelet treatment with either 1 U and 10 U pertussis toxin prevented chemotaxis to fMLF compared with platelets not treated with pertussis toxin. In panels b and c, platelets were activated with 10 U thrombin. Results are expressed as the mean value (±SD) of migrated cells, in triplicate samples, of five independent experiments.

FIG. 5 shows chemotaxis of human platelets in response to endogenous danger signals. Human platelets treated with 10 U thrombin were seeded upon a two micron pore membrane and allowed to migrate towards the indicated chemoattractant placed in the lower chamber of a transwell dish. a, Thrombin-activated platelets showed significant migration towards whole and lysed mitochondria compared with golgi. Platelets also showed significant migration toward lysed mitochondria compared with whole mitochondria. Platelet chemotaxis in response to whole and lysed mitochondria was significantly inhibited by platelet treatment with 100 μg α-FPR, compared with isotype-matched antibody, IgG (100 μg). b, Thrombin-activated platelets showed significantly more migration toward necrotic cell supernatant, derived either by repeated freeze/thawing or UV (1000 J) exposure, compared with apoptotic cell supernatant. This migration was blocked by pretreatment of the platelets with 100 μg α-FPR, however migration was unaffected by isotype specific antibody, IgG (100 μg). c, Thrombin-activated platelets showed significantly more migration towards ischemic human aortic endothelial cells compared with non-ischemic human aortic endothelial cells. Migration was inhibited by platelet pretreatment with α-FPR, however migration was unaffected by isotype specific antibody, IgG. d, Human platelet migration toward ischemic human aortic endothelial cells was inhibited by pertussis toxin treatment of thrombin-activated platelets. Platelets were activated with 10 U thrombin. Results are expressed as the mean value (±SD) of migrated cells, in triplicate samples, of 3 independent experiments.

### DETAILED DESCRIPTION OF THE INVENTION

The present invention relates to immunogenic peptides, variants, derivatives, or analogs thereof, from N-formyl-modified peptides. N-formyl peptides may be obtained from any source, e.g. either isolated from natural or recombinant sources or produced synthetically. For example, the peptides may be derived from bacteria, eukaryotic mitochondria, or synthesized. More specifically, peptides derived from N-formyl-modified proteins bind to specific receptors on phagocytic cells and thereby stimulate an immune response, inducing directed migration or chemotaxis. Further, the present invention relates to methods of using N-formyl peptides to mobilize platelets to an injury site and methods of using N-formyl peptide receptor antagonists, such as formyl peptide specific antibodies or other small molecule antagonists, to suppress an immune response and more specifically, inhibit inflammation at an injury site.

An “immunostimulatory peptide” is defined herein as a peptide which is modified at the amino terminal end with a formyl group and is capable of causing a cellular or humoral immune response in a mammal.

Embodiments of the present invention relate to isolated N-formyl peptides. The N-formyl peptide further refers to the amino acid sequence of substantially purified N-formyl polypeptide, which may be obtained from any species, preferably mammalian, and more preferably, human, and from a variety of sources, including natural, synthetic, semi-synthetic, or recombinant. Functional fragments of the N-formyl polypeptide are also embraced by the present invention. N-Formyl peptides are short peptides which may be generated by an endopeptidase cleavage of the first few amino acids including the N-formyl-modified methionine group of proteins²⁸. Alternatively, the N-formyl peptides of the invention may be synthetically manufactured or produced recombinantly in an adopted host or organism. In short, the N-formyl peptides of the invention include N-formyl peptides derived from any source. The N-formyl peptides are preferably 2-50 amino acids in length, more preferably 3-10 amino acids in length.

In a preferred embodiment of the present invention, both endogenous and exogenous N-formyl peptides induce an immune response, where platelets migrate towards a gradient of endogenous and exogenous N-formyl peptides using N-formyl peptide receptors. N-Formyl peptides are commonly known to bind to specific receptors on phagocytic cells, and induce directed migration or chemotaxis²⁹⁻³¹. “Chemotaxis” is defined herein as a response of mobile cells, in which the specific direction of movement or mobilization is affected by a gradient, such as of N-formyl peptides through their specific N-formyl peptide receptor; whereas, “chemokinesis” is defined as stimulated motility that is random in direction. For the first time, human platelets have been demonstrated to migrate towards a gradient of both endogenous and exogenous N-formyl peptides using functional N-formyl peptide receptors. The significance of this finding is that platelets have unique immunostimulatory properties (known as costimulatory properties) that are likely to augment most immune responses, and thus be fundamental to the establishment of most adaptive and counter-adaptive immune responses.

In addition to chemotaxis, the N-formyl peptide/receptor interaction also stimulates changes in intracellular calcium. Previous studies have demonstrated that all platelet excitatory agonists except adrenaline have the capacity to induce an increase in cytosolic calcium³⁷. Thrombin-activation of platelets induces a rapid dose-response increase in cytoplasmic calcium^{37,38}. Furthermore, formyl peptide receptor (FPR) or formyl peptide receptor-like 1 (FPRL 1) binding of formyl peptides on phagocytic cells elicits a well-characterized calcium influx^{39,40,41} in addition to chemotaxis.

Chemotaxis occurs, for example, via the commonly known binding of formyl peptides to specific receptors on phagocytic cells, monocytes, or neutrophils. Different types of formyl peptide receptors are also expressed on these types of cells. In one example, human phagocytes express two formyl peptide receptors, FPR (formyl peptide receptor) and FPRL 1 (FPR-like 1), both of which couple to pertussis toxin-sensitive G proteins^{31,33}. FPR has been shown to bind formyl peptides at a 1,000 fold higher affinity than FPRL 1, and has been attributed with inducing chemotaxis. This embodiment of the invention presents platelets expressing N-formyl peptide receptors. Furthermore, this embodiment provides platelets expressing N-formyl peptide receptors that bind N-formyl peptides. Based on chemotactic actions of N-formyl peptides that attract phagocytes to sites of infection and injury and therefore play an important role in microbicidal and other host defense activities^{34,35}, platelets expressing FPRs are similarly attracted to the N-formyl peptide gradient released at sites of injury.

Since N-formyl peptides are well-characterized chemoattractants for phagocytic leukocytes and monocytes^{29,30,31,32}, the presence of functional N-formyl peptide receptors on the surface of activated platelets suggest a functional component in platelets analogous to activated phagocytes. More specifically, this embodiment provides antibodies raised against the N-formyl peptide receptor that block the function of N-formyl peptide receptors.

In a further embodiment, the present invention relates to an antibody having affinity for N-formyl peptides or peptide fragments thereof. The invention also relates to binding fragments of such antibodies. In one preferred embodiment, the antibodies are specific for N-formyl peptides, where these N-formyl peptides bind to receptors expressed on platelets. Antibodies are preferably raised to N-formyl peptides or fragment peptides, either naturally-occurring or recombinantly produced, using methods well known in the art.

The N-formyl peptides of the present invention may be readily prepared by techniques, including, but not limited to the Merrifield solid-phase peptide synthesis technique commonly known in the art and described, for example, by Steward and Young, “Solid Phase Peptide Synthesis” (W.H. Freeman & Co., San Francisco, 1969). Formylation of the synthesized peptide may be carried out by the method of Sheehan and Yang (J. Am. Chem. Soc., Vol. 80, p. 1154, 1958).

The N-formyl peptides and peptide fragments thereof described above may be joined to other materials, particularly polypeptides, as fused or covalently joined polypeptides to be used as carrier proteins. In particular, N-formyl peptides or fragments can be fused or covalently linked to a variety of carrier proteins, such as keyhole limpet hemocyanin, bovine serum albumin, tetanus toxoid, etc. See for example, Harper and Row, (Microbiology, Hoeber Medical Division, 1969); Landsteiner, Specificity of Serological Reactions (Dover Publications, New York, 1962); and Williams et al., (Methods in Immunoloay and Immunochemistry, Vol. 1 Academic Press, New York, 1967), for descriptions of methods of preparing polyclonal antisera. A typical method involves hyperimmunization of an animal with an antigen. The blood of the animal is then collected shortly after the repeated immunizations and the gamma globulin is isolated.

In some instances, it is desirable to prepare monoclonal antibodies from various mammalian hosts. Description of techniques for preparing such monoclonal antibodies may be found in Stites et al., (Basic and Clinical Immunology, Lange Medical Publications, Los Altos, Calif., Fourth edition) and references cited therein, and in particular in Kohler and Milstein (Nature 256:495-497, 1975) which discusses one method of generating monoclonal antibodies.

The danger model of immunity demonstrates that APCs and subsequent T cell activation is dependent upon detection of danger signals released by injured or necrotic tissues or cells. Blocking N-formyl peptide signaling with an inhibitor, or preferably a neutralizing antibody against the N-formyl peptide receptor may provide a means of attenuating such danger signals elicited by inflammatory processes.

In another embodiment, a method of inhibiting an immune response comprises administering an effective amount of N-formyl peptide receptor inhibitor, preferably an antibody directed against an N-formyl peptide receptor or a peptide with competitive inhibitory properties. The FPR antibody blocks the function of the FPR, thereby inhibiting the N-formyl peptide/FPR interaction.

In a further embodiment, a method of blocking inflammation at a site of injury comprises administering an effective amount of N-formyl peptide receptor inhibitors or more preferably antibodies to block inflammation or counter adaptive immune reactivity. Preferably, the N-formyl peptide receptor antibody is used to block inflammation in the treatment of chronic and acute inflammatory diseases, conditions, and syndromes such-as immune diseases (e.g systemic lupus erythematosus, allograft rejection).

FPR antibodies are preferably administered to block the inflammation process as well as other immune-related responses and thus treat a subject having an overactive immune response. For example, a subject having acute respiratory distress syndrome has an overabundance of neutrophils which is representative of an overactive immune response. FPR antibodies are preferably administered in an effective amount to the subject in order to inhibit the immune response and preferably block inflammation in order to treat the subject having acute respiratory distress syndrome. Other non-limiting examples of diseases, conditions, or syndromes treated with FPR antibodies or inhibitors include: acute organ or tissue transplant rejection, rheumatoid arthritis, systemic lupus erythematosus, multiple sclerosis, immune activation resulting from traumatic injuries, and the inflammatory sequelae of chronic or acute infections or traumatic injury or injury secondary to toxin exposure.

In a further embodiment, the present invention relates to a method of mobilizing platelets via N-formyl peptide receptors. Since endogenous or exogenous N-formyl peptides bind the N-formyl peptide receptors expressed on platelets, chemotaxis or the mobilization of platelets occurs. Furthermore, injured tissues or cells release N-formyl peptides. In this manner, platelets may be directed to and used in the treatment of injuries where platelets are particularly useful for their blood coagulation and hemostasis properties. Preferably, a method of mobilizing platelets to an injury site comprises administering an effective amount of N-formyl peptides, preferably at the site of injury.

More specifically, a method of mobilizing platelets to cells undergoing necrotic cell death or ischemia or reperfusion due to cell injury is provided. Platelet chemotaxis was observed in the context of necrotic cell death by simulating such conditions, including but not limited to freeze/thaw and UV irradiation. Significant platelet chemotaxis was induced and was further inhibited by anti-FPR blockade. However, apoptotic cells did not induce platelet chemotaxis. Therefore, the method of platelet mobilization using N-formyl peptides of the invention specifically targets cells undergoing necrotic cell death or ischemia. In particular, a method of mobilizing platelets to cells undergoing necrotic cell death or ischemia or reperfusion due to cell injury comprising administering an effective amount of N-formyl peptides is provided.

These observations suggest that endogenous N-formyl peptides released with cell injury and necrosis provide a danger signal to myeloid elements of the innate immune response. Moreover, they provide a mechanism for localizing CD154-bearing platelets to sites of cellular and tissue injury, thus allowing for their engagement with APC. Therefore, in addition to platelets, other FPR-bearing myeloid cells including neutrophils and monocyte/macrophages, may utilize a similar mechanism for identifying and responding to tissue injury. Further, immature dendritic cells express FPR and may provide a means of maintaining residence in dying tissue by binding endogenous formyl peptides⁴³. With activation and maturation, however, dendritic cells lose FPR expression and migrate to draining lymph nodes for T cell activation⁴⁴. Thus, one of the most primitive and ubiquitous signaling peptides may provide the basis of the complex and well-orchestrated dendritic and T-cell response. Potentially, blockade of platelet or phagocytic cell migration in the context of tissue injury may allow abrogation of one of the earliest signals in the immune response. Therefore one embodiment of the invention relates to the therapeutic application of FPR antibodies in the prevention of counter adaptive inflammation for treatment of conditions such as, for example, acute conditions as trauma and the resultant multisystem organ failure, acute respiratory distress syndrome as a result of severe trauma or toxic inhalation injuries, stroke, myocardial infarction, and solid organ transplant (allograft and xenograft) rejection, reperfusion injury following limb replantation, intestinal ischemia. Examples of therapeutic use of FPR antibodies for attenuation of counter adaptive inflammatory responses in chronic inflammation include vasculitis (autoimmune), arthritis (autoimmune and chronic infections), and multiple sclerosis.

In yet another embodiment of the present invention, therapeutic methods for wound healing, particularly in burn patients or victims of chemical or thermal injury are provided. Preferably, the method for wound healing comprises administering N-formyl peptides to a subject in need thereof, in an effective amount to induce platelet chemotaxis to the wound and/or site of injury. In so doing, platelets provide critical factors essential for blood coagulation and wound repair at the site of injury.

Any of the therapeutic methods described above may be applied to any subject or individual in need of such therapy, including, but not limited to, for example, mammals such as dogs, cats, cows, horses, rabbits, monkeys, and most preferably, humans.

In a further embodiment of this invention, the recombinant or natural N-formyl protein, peptides, or analogs thereof, and/or pharmaceutical compositions or formulations comprising the recombinant or natural N-formyl protein, peptides, or analogs thereof that are useful for inducing an immune response and more specifically, treating inflammation and mobilizing platelets.

This invention also encompasses proteins or peptides that bear a formyl group on the N-terminal amino acid. The only requirement is a formyl group present on the amino terminal amino acid. No peptide sequence homology is necessary-this invention encompasses all peptide sequences with N-formyl groups.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise N-formyl peptides, antibodies to N-formyl peptide receptors, mimetics, agonists, antagonists, or inhibitors of N-formyl peptides. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a subject alone, or in combination with other agents, drugs, hormones, or biological response modifiers.

The pharmaceutical compositions for use in the present invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the N-formyl peptide, or functional fragments thereof, or N-formyl peptide receptor antibodies), the pharmaceutical compositions may contain suitable pharmaceutically acceptable carriers or excipients comprising auxiliaries which facilitate processing of the active compounds into preparations which can be used pharmaceutically. The carrier must also be compatible, where the term “compatible”, as used herein, means that the components of the pharmaceutical compositions are capable of being commingled with small peptides and/or antibodies directed to the small peptides of the present invention, in such a manner such that does not substantially impair the desired pharmaceutical efficacy. Further details on techniques for formulation and administration are provided in the latest edition of Remington's Pharmaceutical Sciences (Mack Publishing Co., Easton, Pa.).

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model may also be used to determine the appropriate concentration range and route of administration. Such information can then be used and extrapolated to determine useful doses and routes for administration in humans. Various concentrations may be used in preparing compositions incorporating the same ingredient to provide for variations in the age of the subject, the severity of the condition, and the duration of the treatment, and the mode of administration.

A therapeutically effective dose refers to that amount of active ingredient, for example, N-formyl peptides, or fragments thereof, antibodies to N-formyl peptide receptors, agonists, antagonists or inhibitors of N-formyl peptide receptors, which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, ED₅₀/LD₅₀. Pharmaceutical compositions, which exhibit large therapeutic indices, are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The exact dosage will be determined by the practitioner, who will consider the factors related to the individual requiring treatment. Dosage and administration are adjusted to provide sufficient levels of the active moiety or to maintain the desired effect. Factors, which may be taken into account, include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

Doses of the pharmaceutical compositions will vary depending upon the subject and upon the particular route of administration used. The dosage will further depend on the method of use of the N-formyl peptide composition. In particular, dosages for administering the pharmaceutical composition comprising N-formyl peptides or fragments thereof for the mobilization of platelets to a site of injury range from 10 to 40,000 μg/kg/day, more preferably 1 to 20 mg/kg/day, and most preferably 5 to 10 mg/kg/day. Doses are typically administered from once a day to every 4 to 12 hours depending on the severity and route of administration of the condition. For acute conditions, it is preferred to administer the N-formyl peptide or composition every 8 to 12 hours. For maintenance or therapeutic use, it may be preferable to administer only once or twice a day. Preferably, from about 1 to about 10 mg/kg of peptide are administered per day, depending on the route of administration and severity of the condition.

In another embodiment, dosages for administering the pharmaceutical composition comprising N-formyl peptide receptor antibodies or inhibitors for inhibiting an immune response, more particularly, blocking inflammation, range from 10 to 40,000 μg/kg/day, more preferably 1 to 20 mg/kg/day, and most preferably 5 to 10 mg/kg/day. Doses are typically administered from once a day to every 12 to 24 hours depending on the severity and route of administration of the condition. For acute conditions, it is preferred to administer the N-formyl peptide receptor antibody or inhibitor or composition every 12 to 24 hours. For maintenance or therapeutic use, it may be preferable to administer only once or twice a day. Preferably, from about 5 to about 20 mg of peptide/kg are administered per day, depending on the route of administration and severity of the condition.

Pharmaceutical compositions for oral administration may be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use may be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of N-formyl peptide or N-formyl peptide receptor antibody product, such labeling would include amount, frequency, and method of administration.

All books, articles, and patents referenced herein are incorporated by reference in toto. The following examples illustrate various aspects of the invention and in no way intended to limit the scope thereof.

### EXAMPLE 1

### Expression of Formyl Peptide Receptors on Human Platelets

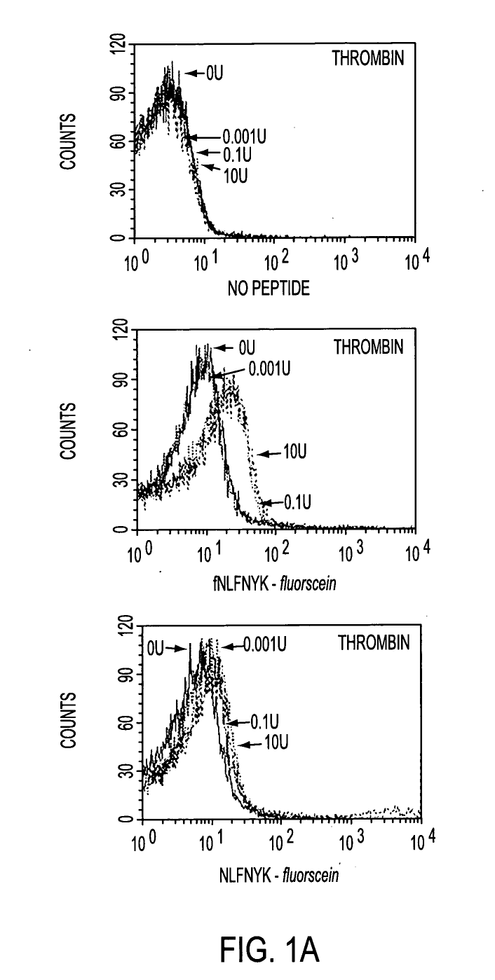

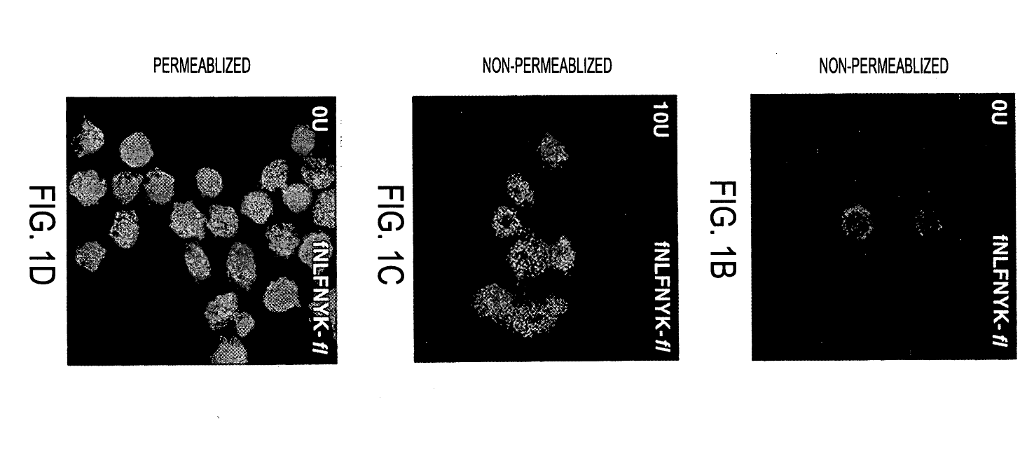

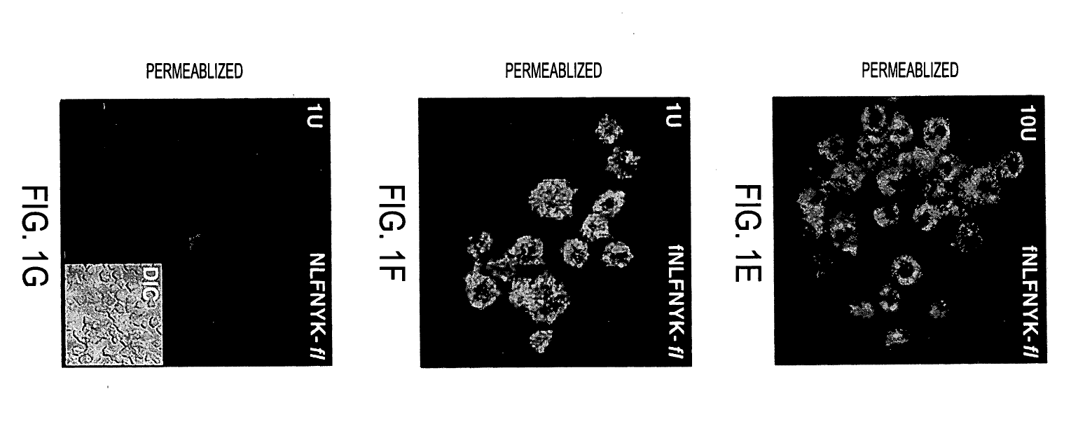

To test whether human platelets express N-formyl peptide receptors, the binding of identical fluorescein-conjugated peptides to human platelets was evaluated by flow cytometry, where one contained a formyl group N-formyl-Nle-Leu-Phe-Nle-Tyr-Lys (fNLFNYK-fl) (SEQ ID NO:1), and the other without a formyl group Nle-Leu-Phe-Nle-Tyr-Lys (NLFNYK-fl) (SEQ ID NO:2). N-Formyl-NLFNYK-fl (SEQ ID NO:1) (10⁻⁷ M) bound to thrombin-activated platelets, showing increased binding with increased thrombin activation (FIG. 1a, middle panel). Conversely, binding of NLFNYK-fl (SEQ ID NO:2) was non-specific and did not increase with thrombin activation (FIG. 1a, lower panel). Corroborating these findings, confocal microscopy using fNLFNYK-fl (SEQ ID NO:1) (10⁻⁶ M) demonstrated upregulation of external N-formyl peptide binding with platelet activation (non-permeablized/10 U thrombin, FIG. 1c) compared with non-permeablized, non-activated platelets (FIG. 1b). However, with permeabilization of non-activated platelets, fluorescein staining of granule-like particles was detected within the interior of platelets (FIG. 1d). Furthermore, with thrombin activation, platelet permeabilization showed that the granule-like distribution of fNLFNYK-fl (SEQ ID NO:1) binding had mobilized to the platelet surface (FIG. 1e). Thrombin-activated platelets showed no binding of non formyl-NLFNYK-fl (SEQ ID NO:2) by confocal microscopy (FIG. 1g) compared with similarly activated platelet binding of fNLFNYK-fl (FIG. f). Differential interference contrast (DIC) imaging (FIG. 1g insert) confirmed the presence of platelets.

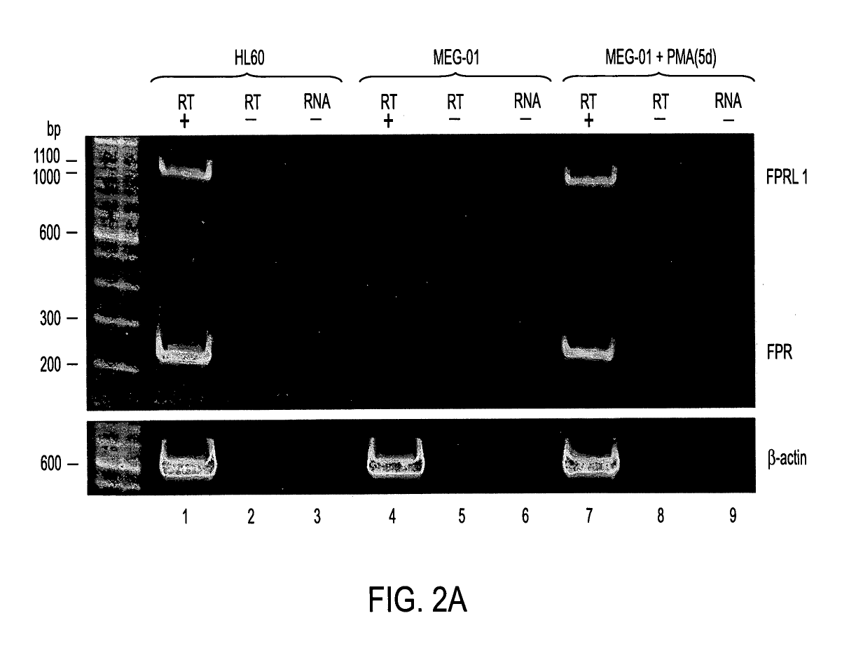

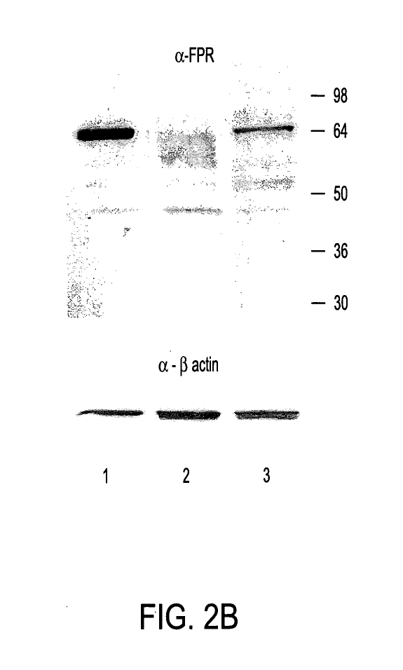

To further confirm that platelets express human N-formyl peptide receptors (FPR), RT-PCR of RNA harvested from the megakaryocytic cell line MEG-01 was performed. Platelets were anucleated, thus indicating that their protein content was a consequence of gene expression in precursor megakaryocytes. Treatment of MEG-01 cells with phorbol 12-myristate 13-acetate (PMA) induced differentiation with production of platelet-like structures³⁶. RNA harvested from HL60 cells, a promyelocytic cell line that expresses both FPR and FPRL 1, is shown in lane 1 (RT included), lane 2 (RT excluded), and lane 3 (RNA excluded) (FIG. 2a). Similarly treated, undifferentiated MEG-01 cell RNA, shown in lane 4 (RT included), lane 5 (RT excluded), and lane 6 (RNA excluded) (FIG. 2a), showed no FPR mRNA. Induction of megakaryocyte differentiation with PMA resulted in transactivation of the genes encoding both the FPR and FPRL-1, shown in lane 7 (RT included), lane 8 (RT excluded), and lane 9 (RNA excluded) (FIG. 2a), as detected by RT-PCR following five days of PMA treatment. RT-PCR generated bands were confirmed to encode the FPR or FPRL1 by sequence analysis. FPR protein expression in platelets was shown by immunoblotting with FPR specific antibody. Expression of FPR was detected in platelets (FIG. 2b, lane 3) and in differentiated CD34+cells (FIG. 2b, lane 1) but not in HEK 293 cells transfected to express FPRL 1 (FIG. 2b, lane 2), thus showing specificity of the antibody to FPR.

### EXAMPLE 2

### Immunofluorescence Staining/Flow Cytometry

Platelet-rich plasma was prepared from whole blood by differential centrifugation at 800×g for 5 min at 22° C. The top two-thirds of the platelet-rich plasma was removed and washed in PBS containing 1% fetal bovine serum. Washed platelets were activated with 0, 0.001, 0.1 or 10 U thrombin (Sigma, St. Louis, Mo.) for 10 min at 37° C. The platelets were then washed and incubated with 10⁻⁷ M fluorescein conjugated N-formyl peptide; fNLFNYK-fl (SEQ ID NO:1; Molecular Probes, Eugene, Oreg.) or 10⁻⁷ M fluorescein conjugated non-N-formylated peptide; NLFNYK-fl (SEQ ID NO:2; New England Peptide, Fitchburg, Mass.) for 30 min at room temperature. The platelets were washed two times with PBS+1% FBS and fixed with 4% paraformaldehyde (Electron Microscopy Sciences, Ft. Washington, Pa.). Samples were analyzed on a Becton Dickinson (Franklin Lakes, N.J.) FASCcalibur/CELLQuest system. Representative traces from at least four independent experiments are shown.

### EXAMPLE 3

### Confocal Microscopy

Platelet-rich plasma was prepared as mentioned above. Washed platelets were activated with either 1 or 10 U thrombin (Sigma) for 10 min at 37° C. The platelets were washed two times and fixed with 4% paraformaldehyde for 20 min at 4° C. The platelets were then washed and permeablized with 100% methanol for 15 min at 4° C. Following two washes, platelets were stained with 10⁻⁷ M fNLFNYK-fl (SEQ ID NO:1; Molecular Probes) or 10⁻⁷ M non-formyl peptide NLFNYK-fl (SEQ ID NO:2; a non-N-formylated version of fNLFNYK-fl from Molecular Probes, generated by New England Peptide, Fitchburg, Mass.) for 30 min at 4° C. The platelets were washed two times, spun onto cover slips, and mounted with ProLong® Antifade Kit (Molecular Probes). Confocal images were collected on a Leica TCS-NT/SP confocal microscope (Leica Microsystems, Exton, Pa.) using a 100-x oil emersion objective NA 1.4, zoom 2. Representative images from three independent experiments are shown (FIG. 2).

### EXAMPLE 4

### Reverse Transcription-Polymerase Chain Reaction

Total RNA was isolated using Ultraspec RNA isolation system (Biotecx Laboratories Inc., Houston Tes.), according to the manufacturer's protocol. To confirm purity of the product RNA, absorption ratios at 260/280 nm were determined to be >1.0 for all samples. The samples were adjusted to 200 ng/μl for reverse transcription and PCR according to absorption at 260 nm. Total RNA was reversed-transcribed using Thermoscript RT (GibcoBRL, Gaithersburg, Md.). Conditions for PCR amplification of the resulting first-strand DNA template involved preheating a mixture of Taq DNA polymerase (5 U/ml) (GibcoBRL), primers, cDNA, and PCR components to 95° C. for 5 min before amplification. Primers for human FPR and human FPRL-1 were generated from sequences reported on Genbank. Human FPR: sense primer 5′-GTCTCCAGTTGGACTAGCCAC-3′ (SEQ ID NO:3); antisense primer 5′-AATGTCCTCCCATGGCCTTCC-3′ (SEQ ID NO:4). Human FPRL-1: sense primer 5′-GCTCTGGCTGTGCATTCAGCAGATT-3′ (SEQ ID NO:5); antisense primer 5′-AAAAGTCAGCCAGGGCCAGGTTACG-3′ (SEQ ID NO:6). The PCR cycle consisted of 20 sec at 95° C. (dissociation), 20 sec at 56° C. (annealing) for FPR and 20 sec at 58° C. for FPRL-1, and 30 sec at 72° C. (extension) Amplification was within the exponential range for all of the primers used. The constitutively expressed “housekeeping” gene β-actin (Stratagene; La Jolla, Calif.) was used as a reference gene for normalization. PCR products were sequenced and found to be greater than 97% identical to the published sequence (FPR: M84562; FPRL-1: L10820).

### EXAMPLE 5

### Western Blot Analysis

Western blot analysis was performed on total protein lysates prepared in 1% SDS-Page sample buffer, heated to 90° C. for 10 min and subjected to gel electrophoresis in 20% Tris-glycine gels (Novel Experimental Technologies (NOVEX; San Diego, Calif.). CD34+ cells were differentiated into neutrophils by incubation with rhIL-3, rhIL-6, rhSCF, rhGM-CSF, rhIL-1β, and rhG-CSF for 21 days in X-VIVO medium supplemented with 1% human serum albumin. Proteins were transferred to nitrocellulose membranes (NOVEX) and probed with antibodies. FPR antibody (PharMingen; San Diego, Calif.) was used at a 1:50 dilution. Following incubation with the secondary antibody, peroxidase labeled sheep anti-mouse IgG (Amersham; Buckinghamshire, England) (1:1000) the blot was developed using the enhanced chemiluminescence (ECL) reagents from Amersham (Buckinghamshire, England). Approximately equal loading of each lane was confirmed by using a mouse monoclonal antibody to actin (1:1000) (Boehringer Mannheim; Indianapolis, Ind.).

### EXAMPLE 6

### Chemotactic Activity of Platelets

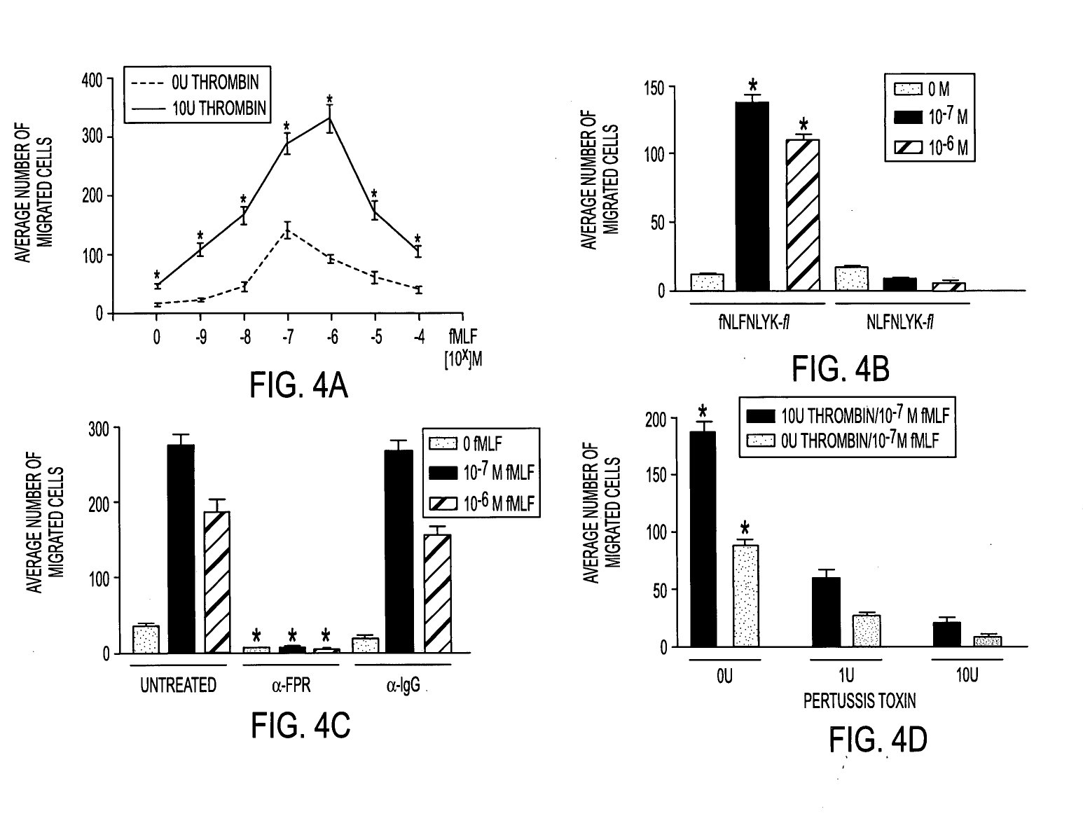

The chemotactic activity of activated and non-activated platelets in response to different concentrations of fMLF by transwell migration (FIG. 4a) was determined. Platelets activated with thrombin demonstrated significant chemotaxis to N-formyl peptides compared with non-activated platelets (P<0.001). Both thrombin-activated and non-activated platelet migration followed a concentration-response curve that was greatest at 10⁻⁶ M fMLF (27.2±12) in the thrombin-activated platelets and 10⁻⁷ M fMLF (11.5±6.5) in the non-activated platelets (FIG. 4a). Thus, platelet chemotaxis occurred with similar N-formyl peptide dose response kinetics as seen with the chemotaxis of phagocytic cells⁴². Next the dependence of platelet chemotaxis on the presence of a Formyl group on the peptide was tested. Non formyl peptide, NLFNYK-fl (SEQ ID NO:2), at both 10⁻⁶ and 10⁻⁷ M concentrations, failed to induce significant chemotaxis of activated platelets compared with N-formyl peptide (P<0.001) (FIG. 4b). Non-activated platelets behaved similarly. Thus, the presence of a Formylgroup was essential for FPR binding, calcium mobilization and platelet chemotaxis.

In order to further confirm the specificity of the chemotactic response to N-formyl peptide binding of the FPR, platelet FPR was inhibited by several methods to establish its functional requirement for platelet chemotaxis. Platelets were pretreated with an FPR-specific antibody (α-FPR) capable of blocking the binding of N-formyl peptides to the FPR receptor, prior to the transwell migration assay. Treatment of platelets with α-FPR blocked chemotaxis of thrombin-activated platelets compared with platelets treated with isotype specific antibody (P<0.001) (FIG. 4c). Additionally, platelets were treated with pertussis toxin, a specific inhibitor of the a-subunit of members of the G_{i/o} class of G-proteins required for FPR activation. Treatment with pertussis toxin significantly decreased migration of both non-activated and thrombin-activated platelets, at both 1 U (P<0.001) and 10 U (P<0.001) compared with platelets that were not treated with pertussis toxin (FIG. 4d). These experiments demonstrated the requirement of FPR on activated platelets for platelet chemotaxis to N-formyl peptides.

### EXAMPLE 7

### Measurement of Chemotaxis

Platelet migration was assessed using a 96-well ChemoTx® microplate (Neuro Probe, Gaithersburg Md.). For all chemotaxis experiments, different concentrations of stimulants were placed in the bottom wells of the microplate and the filter was positioned (2 μm pore size). The cell suspension was seeded onto the top of the filter (30 μl) and the microplates were incubated for 2 hrs at 37° C. For all chemokinesis experiments, different concentrations of stimulants were placed in the bottom wells of the microplate and the filter was positioned. The cell suspension containing different concentrations of stimulants were placed on the filter (30 μl) in a gradient fashion. After incubation, the lid was removed and the filter was gently wiped with a cell harvester and carefully flushed with media to remove any of the non-migrated cells. The filter was then carefully removed and the migrated cells were counted by light microscopy. Results are expressed as the mean value (±SD) of total migrated cells, in triplicate samples, and are taken from five experiments independent.

Chemotactic responses were distinguished from chemokinetic responses by placing increasing concentrations of fMLF in the upper and lower chambers of the transwell plates (Table 1). The activity that has been defined is directional, because “checkerboard assays” (see Zigmond and Hirsch, 1973, J. Exp. Med. 137:387-410) showed that lymphocytes migrated when chemoattractant was present in the bottom chamber and not in the top, but that migration fell off as chemoattractant was added to the top chamber.

Checkerboard analysis showed that significantly more platelets migrated when higher concentrations of fMLF were present in the lower wells of the chemotactic plate relative to the upper wells (Shaded results, Table 1; P<0.002). There was no enhanced cell migration when higher concentrations of fMLF were present in the upper wells or with equal concentrations of fMLF in both the upper and lower wells, as would be seen with chemokinesis. Maintaining the concentration gradient across the transwell membrane permitted chemotaxis to occur; establishing that platelet movement was the consequence of fMLF induced directed migration and not chemokinesis.

TABLE 1
CHECKERBOARD ANALYSIS OF
PLATELET MIGRATION IN RESPONSE TO FMLF

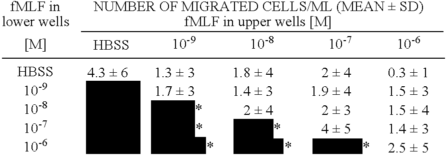

Different concentrations of fMLF were placed in the upper and/or lower wells of the chemotaxis plate; platelets at 10⁶ cells/ml were placed on the upper filter. After a 2-hour incubation, the non-migrated cells were removed and the cells that migrated across the filter were counted. The results are expressed as the mean value (±SD) of the migrated cells in at least six separate experiments (n=24). Grey shading indicates wells containing a greater concentration of fMLF in the lower well compared to the upper well. Platelet migration in transwells maintaining a gradient of fMLF between chambers were compared with wells containing equimolar concentrations of fMLF in both chambers (p<0.002, Student's t-test)

### EXAMPLE 8

### Endogenous Platelet Chemotaxis

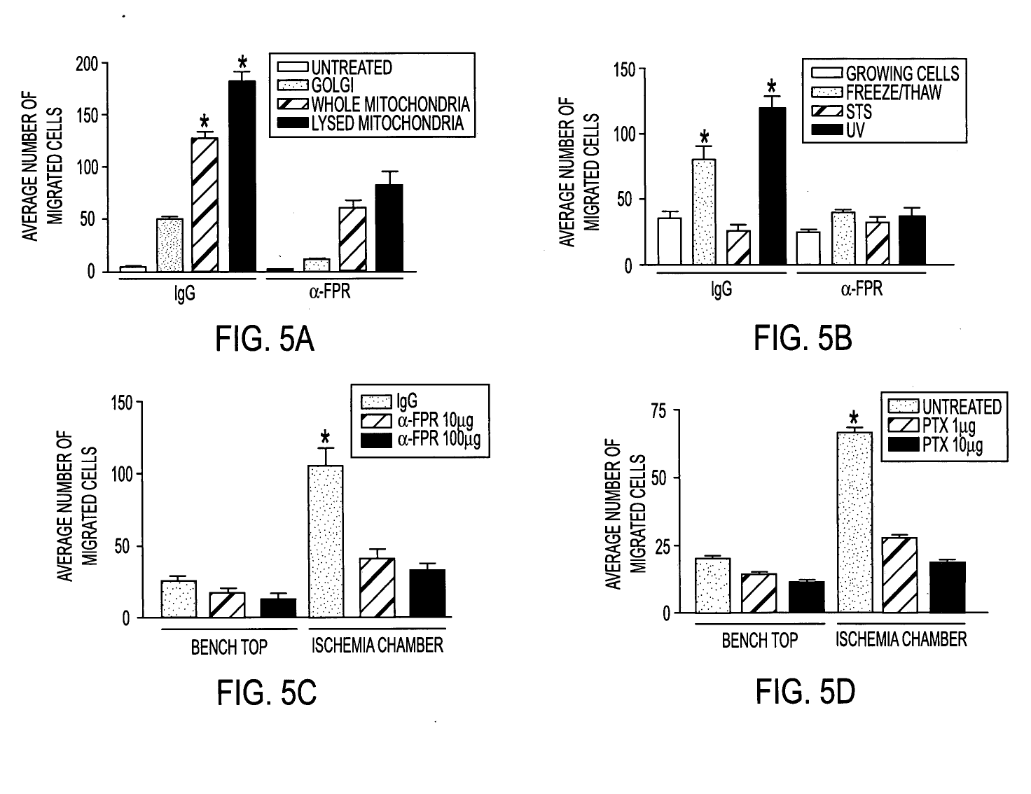

To test platelet chemotaxis towards an endogenous source of N-formyl peptides, mitochondria (whole or lysed by sonication) or golgi (a non N-formyl peptide control) were isolated from Hep-2 cells and placed in the lower chambers of the transwell plates. Thrombin-activated platelets showed significant chemotaxis towards lysed mitochondria (whole or lysed by sonication) or golgi (a non N-formyl peptide control) isolated from Hep-2 cells and placed in the lower chambers of the transwell plates. Thrombin-activated platelets showed significant chemotaxis towards lysed mitochondria compared with either whole mitochondria or golgi (FIG. 5a; P<0.001). Non-activated platelets also showed significant chemotaxis towards both lysed mitochondria and whole mitochondria compared to golgi. Mitochondria were previously shown to be a source of N-formyl peptides³⁰⁻³⁵, Bianchetti, R., Lucchini, G., Sartirana, M. L. (1971) Endogenous synthesis of formyl-methionine peptides in isolated mitochondria and chloroplasts. Biochemical and Biophysical Research Communications; 42:97-102, and suggest that platelet chemotaxis may be due to mitochondrial components other than N-formyl peptides. Unfortunately, neutralizing antibody against N-formyl peptides themselves does not exist; hence it is not possible to test the specificity of this response by blocking N-formyl peptides. N-Formyl peptides, however, are the only known ligands of the FPR, unlike FPRL-1 which binds both protein and lipid agonists with high affinity. Treatment of platelets with α-FPR blocked chemotaxis of both non-activated and thrombin-activated platelets to mitochondria compared with platelets treated with isotype specific antibody (FIG. 5a). Thus, blockade of platelet chemotaxis towards mitochondrial proteins with neutralizing antibody against the FPR implicates mitochondrial N-formyl peptides as the chemoattracting agent.

### EXAMPLE 9

### Endogenous Platelet Chemotaxis in Necrotic Cells

Hep-2-cells were grown in DMEM medium supplemented with 10% FCS. Before each experiment, the cells were washed and the media was replaced with serum-free DMEM. After adaptation to this medium, cells were exposed to repeated freeze/thaw or UV radiation (1000J) in a UV Stratalinker UV oven (Stratagene) to induce necrosis. To induce apoptosis, cells were treated with 1 μM staurosporine (STS) for 5 hours. After the treatments, the cells were placed into a tissue culture incubator overnight. The supernatant was collected and used for experimentation and the cells were stained with a mixture of the membrane permeant dye H-33342 (500 ng/ml) and the membrane impermeant dye SYTOX (500 nM) (Molecular Probes; Eugene, Oreg.). Necrotic (damaged plasma membrane; non-condensed nuclei) and apoptotic (intact plasma membrane; characteristically condensed or fragmented nuclei) cells were scored. Necrotic cells after UV radiation=84%. Apoptotic cells after STS treatment=80%.

Platelet chemotaxis was examined in the context of necrotic or apoptotic cell death. Supernatants from cells injured by repeated freeze and thawing, or by UV irradiation, induced significant platelet chemotaxis that was inhibited by α-FPR blockade (FIG. 5b; P<0.0001). Supernatants from cells subjected to apoptotic death using staurosporine (STS) did not elicit platelet chemotaxis (FIG. 5b). Platelets incubated with STS treated cell supernatants, or even STS directly were capable of subsequent calcium flux responses to thrombin and thus were not “poisoned” by the exposure.

### EXAMPLE 10

### Endogenous Platelet Chemotaxis in Ischemia/Reperfusion Cell Injury

Primary human aortic endothelial cells were plated in 0.2% gelatin (Sigma) coated 96-well ChemoTx microplates and allowed to recover for 48 hours. Before the plate was placed in the modular incubator chamber (Billups-Rothenberg Inc.; Del Mar, Calif.) the media was replaced with serum-free media containing 10 mM Hepes. The plate was placed on ice and put into the air tight modular incubator chamber and flushed with a gas mixture of 5% CO₂ and 95% N₂ for 90 min to induce ischemia. The cell culture was then returned to a normoxic environment of atmospheric air/5% CO₂ to simulate reperfusion, and allowed to recover for 4 hours in a cell culture incubator. Chemotaxis experiments were then performed.

In particular, studies were performed to determine whether endogenous danger signals released by tissues undergoing ischemia/reperfusion cell injury induced platelet chemotaxis. Primary human aortic cells, plated in a 96-well ChemoTx plate NeuroProbeInc. Gaithersburg, Md. were placed on ice and put into an air tight modular incubator chamber that was flushed with a gas mixture of 5% CO₂ and 95% N₂ for 90 min to induce ischemia. The cell culture was then returned to a normoxic environment of atmospheric air/5% CO₂ and allowed to recover for 4 hours in a cell culture incubator at 37° C. Thrombin-activated platelets showed statistically significant chemotaxis toward aortic endothelial cells, which had undergone ischemia-reperfusion injury compared with untreated cells (FIG. 5c; P<0.001). Treatment of platelets with α-FPR, but not isotype specific antibody, blocked chemotaxis towards injured aortic endothelium (FIG. 5c; P<0.005). Treatment of activated platelets with pertussis toxin also decreased chemotaxis toward ischemic aortic endothelial cells, again supporting specific involvement of the FPR in mediating platelet chemotaxis (FIG. 5d; P<0.0001).

### EXAMPLE 11

### Measurement of Intacellular Free Calcium

Washed platelets were activated with either 0 U or 1 U thrombin (Sigma) for 10 min at 37° C. Fura-2 loaded platelets were prepared by incubating the platelets, for 45 min with fura-2 acetoxymethyl ester at 37° C. The dye-loaded platelets were washed and resuspended in calcium-free HBSS standard solution. The cells were then transferred into quartz cuvettes (10⁹ cells in 2 ml), which were placed in a model MS-III spectrofluorometer (Photon Technologies, Inc.; South Brunswick, N.J.) and continuously stirred at 37° C. Stimulants were added in a 2 μl volume at indicated time points. The ratios of fluorescence at excitation wavelengths 340 and 380 nm and emission wavelength at 510 nm were calculated using FeliX fluorescence analysis software (Photon Technology Instruments; London, Ontario, Canada). Representative traces from at least six independent experiments are shown.

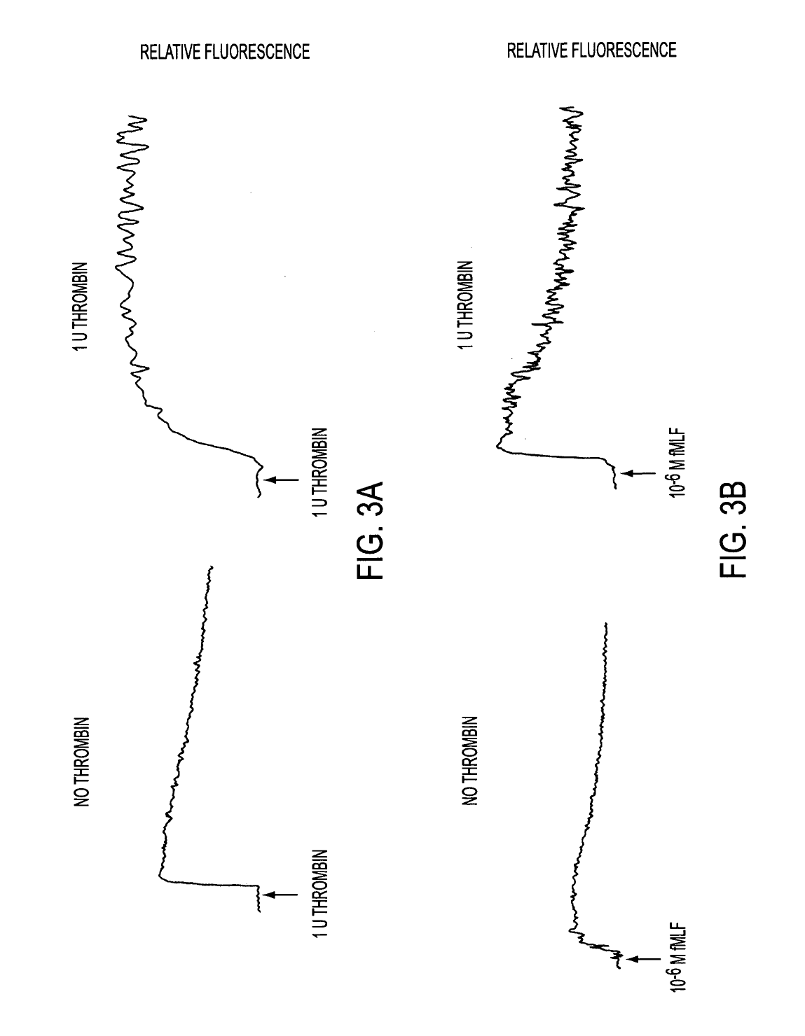

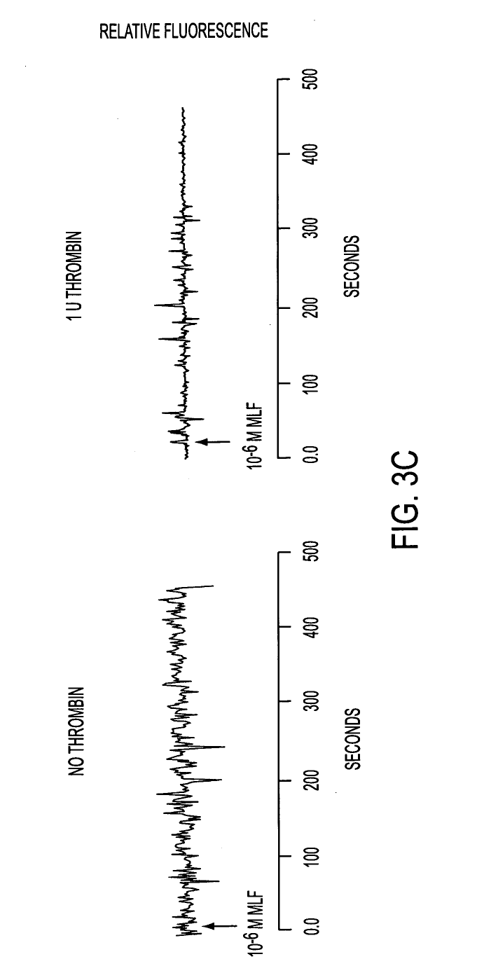

Using fura-2 loaded intact platelets, both thrombin and N-formyl-methionine-leucine-phenalanine (fMLF) induced a calcium signal in thrombin-activated as well as non-activated human platelets (FIGS. 3a and 3b). Consistent with flow cytometry and confocal data, pretreatment with thrombin (1 U), and subsequent fMLF stimulation produced a greater fluorescent signal, supporting FPR upregulation with platelet activation (FIG. 3b). Platelets treated with the non formyl peptide, MLF, either with or without thrombin activation, failed to elicit a calcium signal (FIG. 3c).

### EXAMPLE 12

### Statistical Analysis

All statistical analysis for the chemotaxis experiments was performed using GraphPad InStat software. Student's independent t-tests were performed and all results are expressed as the mean value (±SD) of migrated cells/ml and are taken from at least five independent experiments.

References

1. Matzinger, P. Toleance, danger, and the extended family. Annu.Rev.Immunol. 12, 991-1045 (1994).

2. Gallucci, S., Matzinger, P. Danger signals: SOS to the immune system. Curr.Opin.Immunol. 13, 114-9 (2001).

3. Matzinger, P. An innate sense of danger. Semin.Immunol. 10, 399-415 (1998).

4. Anderson, C. C., Matzinger, P. Danger: the view from the bottom of the cliff. Semin. Immunol. 12, 231-8 (2000).

5. Janeway, C. A. The immune system evolved to discriminate infectious nonself from noninfectious self. Immunol.Today. 13, 11-16 (1992).

6. Fearon, D. T., Locksley, R. M. The instructive role of innate immunity in the acquired immune response. Science 272, 50-53 (1996).

7. Bretscher, P., Cohn, M. A theory of self-nonself discrimination. Science 169, 1042-49 (1970).

8. Janeway, C. A. Approaching the asymptote? Evolution and revolution in immunology. Cold Spring Harb.Syinp.Quant.Biol. 54, 11-13 (1989).

9. Shi, Y., Zheng, W., Rock, K. L. Cell injury releases endogenous adjuvants that stimulated cytotoxic T cell responses. Proc.Natl.Acad.Sci.USA 97, 14590-5 (2000).

10. Larsen, C. P., Elwood, E. T., Alexander, D. Z., Ritchie, S. C., Hendrix, R., Tucker-Burden, C., Cho, H. R., Aruffo, A., Hollenbaugh, D., Linsley, P. S., et al. Long-term acceptance of skin and cardiac allografts after blocking CD40 and CD28 pathways. Nature 381, 434 (1996).

11. Kirk, A. D., Burkly, L. C., Batty, D. S., Baumgartner, R. E., Berning, J. D., Buchanan, K., Fechner, J. H. Jr, Germond, R. L., Kampen, R. L., Patterson, N. B., Swanson, S. J., Tadaki, D. K., TenHoor, C. N., White, L., Knechtle, S. J., Harlan, D. M. Treatment with humanized monoclonal antibody against CD154 prevents acute renal allograft rejection in nonhuman primates. Nat.Med. 5(6), 686-93 (1999).

12. Bingaman, A. W., Ha, J., Waitze, S. Y., Durham, M. M., Cho, H. R., Tucker-Burden, C., Hendrix, R., Cowan, S. R., Pearson, T. C., Larsen, C. P. Vigorous allograft rejection in the absence of danger. J.Immunol. 164(6), 3065-71 (2000).

13. Gallucci, S., Lolkema, M., Matzinger, P. Natural adjuvants: endogenous activators of dendritic cells. Nat.Med. 5, 1249-55 (1999).

14. Sauter, B., Albert, M L., Francisco, L., Larsson, M., Somersen, S., Bhardwej, N. Consequences of cell death: exposure to necrotic tumor cells, but not primary tissue cells or apoptotic cells, induces maturation of immunostimulatory dendritic cells. J. Exp.Med. 191, 423-434 (2000).

15. Krieg, A. M. The role of CpG motifs in innate immunity. Curr.Opin.Immunol. 12, 35-43 (2000).

16. Land, W. Postischemic reperfusion injury and allograft dysfunction: Is allograft rejection the result of a fateful confusion by the immune system of danger and benefit. Transplant.Proc. 31, 332-6 (1999).

17. Todryk, S. M., Melcher, A. A., Dalgleish, A. G., Vile, R. G. Heat shock proteins refine the danger theory. Immunol. 99, 334-7 (2000).

18. Chen, W., Syldath, U., Bellmann, K., Burkart, V., Kolb, H. Human 60-kDa heat-shock protein: A danger signal to the innate immune system. J.Immunol. 162, 3212-19 (1999).

19. Basu, S., Binder, R. J., Suto, R., Anderson, K. M., Srivastava, P. K. Necrotic but not apoptotic cell death releases heat shock proteins, which deliver a partial maturation signal to dendritic cells and activate the NF-kappaB pathway. Int.Immunol. 12, 1539-46 (2000).

20. Cella, M., Scheidegger, K., Palmer-Lehmann, K., Lane, P., Lanzavecchia, A., Alber, G. Ligation of CD40 on dendritic cells triggers production of high levels of interleukin-12 and enhances T cell stimulatory capacity: T-T help via APC activation. J.Exp.Med. 184, 747-752.

21. Gately, M. K., Renzetti, L. M., Magram, J., Stern, A. S., Adorini, L., Gubler, U., Presky, D. H. The interleukin-12/interleukin-12-receptor system: role in normal and pathologic immune responses. Annu.Rev.Immunol. 16, 495-521 (1998).

22. Cella, M., Scheidegger, D., Palmer-Lehmann, K., Lane, P., Lanzavecchia, A., Alber, G. Ligation of CD40 on dendritic cells triggers production of high levels of interleukin-12 and enhances T cell stimulatory capacity: T-T help via APC activation. J.Exp.Med. 184, 747-752 (1996).

23. Hilkens, C. M., Kalinski, P., de Boer, M., Kapsenberg, M. L. (1997). Human dendritic cells require exogenous interleukin-12-inducing factors to direct the development of naive T-helper cells toward the Th1 phenotype. Blood 90, 1920-1926 (1997).

24. Kato, T., Hakamada, R., Yamane, H., Nariuchi, H. Induction of IL-12 p40 messenger RNA expression and IL-12 production of macrophages via CD40-CD40 ligand interaction. J.Immunol. 156, 3932-3938 (1996).

25. Van Kooten, C., Banchereau, J. CD40-CD40 ligand. J.Leuk.Biol. 67, 2-17 (2000).

26. Henn, V., Slupsky, J. R., Grafe, M., Anagnostopoulos, I., Forster, R., Muller-Berghaus, G., Kroczek, R. A. CD40 ligand on activated platelets triggers an inflammatory reaction of endothelial cells. Nature 391, 591-4 (1998).

27. Henn, V., Steinbach, S., Buchner, K., Presek, P., Kroczek, R. A. The inflammatory action of CD40 ligand (CD154) expressed in activated human platelets is temporally limited by coexpressed CD40. Blood 98, 1047-1053.

28. Schiffmann, E., Showell, H. V., Corcoran, B. A., Ward, P. A., Smith, E., Becker, E. L. J.Immunol. 114, 1831-7 (1975).

29. Painter, R. G. et al. Activation of neutrophils by Formylchemotactic peptides. Fed.Proc. 43, 2737-2742 (1984).

30. Carp, H. Mitochondrial N-N-formylmethionyl proteins as chemoattractants for neutrophils. J.Exp.Med. 155, 264-275 (1982).

31. Prossnitz, E. R., Ye, R. D. The N-formyl peptide receptor: a model for the study of chemoattractant receptor structure and function. Pharmacol.Ther. 74, 73-102 (1997).

32. Fiore, S., Maddox, J. F., Perez, H. D., Serhan, C. N. Identification of a human cDNA encoding a functional high affinity lipoxin A4 receptor. J.Exp.Med. 180, 253-260 (1994).

33. Niedel, J. E., Cuatrecasas, P. N-formyl peptide chemotactic receptors of leukocytes and macrophages. Curr.Top.Cell Regul. 17, 137-170 (1980).

34. Murphy, P. M. The N-N-formyl peptide chemotactic response receptor. In Chemoattractant Ligands and their Receptors. R. Hourk, editor. CRC Press, Inc., Boca Raton. Fla. 269-299 (1996).

35. Carp, H. Mitochondrial N-N-formylmethionyl proteins as chemoattractants for neutrophils. J.Exp.Med. 155, 264-275 (1982).

36. Baatout, S. Phorbol esters: useful tools to study megakaryocyte differentiation. Hematol.Cell Ther. 40, 33-39 (1998).

37. Scrutton, M. C. The platelet as a Ca(2+)-driven cell: mechanisms which may modulate Ca(2+)-driven responses. Adv.Exp.Med.Biol. 344, 1-15 (1993).

38. Magocsi, M., Sarkadi, B., Kovacs, T., Gardos, G. Thrombin-induced activation of calcium transport pathways and their role in platelet functions. Biochim.Biophys.Acta. 984, 88-96 (1989).

39. Lew, P. D. Receptors and intracellular signaling in human neutrophils. Am.Rev.Respir.Dis. 141, S127-S131 (1990).

40. Sklar, L. A., Jesaitis, A. J., Painter, R. G. The neutrophil N-formyl peptide receptor: dynamics of ligand-receptor interactions and their relationship to cellular responses. Contemp.Top.Immunobiol. 14, 29-82 (1984).

41. Gao, J. L., Murphy, P. M. Species and subtype variants of the N-formyl peptide chemotactic receptor reveal multiple important functional domains. J.Biol.Chem. 268, 25395-25401 (1993).

42. Hartt, J. K., Barish, G., Murphy, P. M., Gao, J. L. N-N-formyl peptides induce two distinct concentration optima for mouse neutrophil chemotaxis by differential interaction with two N-N-formyl peptide receptor (FPR) subtypes. Molecular characterization of FPR2, a second mouse neutrophil FPR. J.Exp.Med. 190, 741-747 (1999).

43. Yang, D., Chen, Q., Stoll, S., Chen, X., Howard, Z., Oppenheim, J. J. Differential regulation of responsiveness to fMLP and C5a upon dendritic cell maturation: correlation with receptor expression. 2000. J.Immunol. 165, 2694-2702 (2000).

44. Yang, D., Chen, Q., Le, Y., Wang, J. M., Oppenheim, J. J. Differential regulation of N-formyl peptide receptor-like 1 expression during the differentiation of monocytes to dendritic cells and macrophages. J.Immunol. 166, 4092-8 (2001).

What is claimed is:

1. A method of mobilizing platelets at an injury site comprising administering an effective amount of an N-formyl peptide to mobilize said platelets.

2. A method of blocking inflammation at an injury site of a subject in need thereof comprising administering an effective amount of N-formyl peptide receptor inhibitor to block inflammation.

3. The method of blocking inflammation according to claim 2, wherein said N-formyl peptide receptor inhibitor is an N-formyl peptide receptor antibody.

4. A method of blocking inflammation in an organ transplant subject comprising administering an effective amount of N-formyl peptide receptor inhibitor to block inflammation.

5. The method of blocking inflammation according to claim 5, wherein said N-formyl peptide receptor inhibitor is an N-formyl peptide receptor antibody.

6. A method of inhibiting platelet or phagocytic cell chemotaxis comprising administering an effective amount of N-formyl peptide receptor inhibitor to inhibit platelet chemotaxis.

7. The method of inhibiting platelet or phagocytic cell chemotaxis according to claim 6, wherein said N-formyl peptide receptor inhibitor is an N-formyl peptide receptor antibody.

8. A method of treating an inflammatory disease, condition, or syndrome comprising administering an effective amount of N-formyl peptide receptor inhibitor to a subject having the inflammatory disease, condition, or syndrome.

9. The method of treating an inflammatory disease, condition, or syndrome according to claim 8, wherein said inflammatory disease is selected from the group consisting of an acute organ transplant rejection, rheumatoid arthritis, systemic lupus erythematosis, multiple sclerosis, inflammation due to chronic infection, vascular stenosis subsequent to acute myocardial infarction, ischemic/reperfusion injury subsequent to myocardial infarction or stroke, multiple organ failure secondary to trauma, systemic infection, toxin exposure, and autoimmune vasculitis.

10. A method of wound healing comprising administering an effective amount of N-formyl peptide to a subject having a wound to heal the wound.

11. An anti-inflammatory composition comprising an effective amount of N-formyl peptide receptor inhibitor and a pharmaceutical carrier.

12. The anti-inflammatory composition according to claim 11, wherein said N-formyl peptide receptor inhibitor is an N-formyl peptide receptor antibody.

13. A platelet-mobilizing composition comprising an effective amount of N-formyl peptides to mobilize said platelets and a pharmaceutical carrier.

14. A platelet-mobilizing composition comprising an effective amount of an N-formyl peptide having an amino acid sequence of f Nle Phe Nle Tyr Lys to mobilize said platelets and a pharmaceutical carrier.

15. A wound healing composition comprising an effective amount of an N-formyl peptide having an amino acid sequence of f Nle Leu Phe Nle Tyr Lys to heal wounds, and a pharmaceutical carrier.
